(19) United States
(12) Patent Application Publication
(10) Pub. No.: US 2018/0148716 A1
(43) Pub. Date: May 31, 2018

(54) EFFICIENCY IMPROVING LIGATION METHODS

(71) Applicant: QIAGEN GMBH, Hilden (DE)

(72) Inventors: Katja HEITZ, Hilden (DE); Nan FANG, Hilden (DE)

(21) Appl. No.: 15/562,322

(22) PCT Filed: Mar. 31, 2016
(86) PCT No.: PCT/EP2016/057134

§ 371 Date: Sep. 28, 2017

(30) Foreign Application Priority Data
Mar. 31, 2015 (EP) 15162004.4

(51) Int. Cl. C12N 15/10; C12Q 1/6874; C12Q 1/6855

(52) U.S. Cl. CPC C12N 15/1093; C12Q 1/6855; C12Q 1/6874

(57) ABSTRACT

The present invention provides new methods and kits to improve the efficiency of ligation reactions, in particular in molecular biology applications, such as the next generation sequencing (NGS) library construction methods. In next-generation sequencing methods, the ligation step is critical in adding sequencing platform-specific adapters to the DNA fragments that are to be sequenced. Said improvement is achieved by the addition of single- or double-stranded DNA-binding proteins in the ligation step.

### FIELD OF THE INVENTION

The present invention provides new methods and kits to improve the efficiency of ligation reactions, in particular in molecular biology applications, such as the next generation sequencing (NGS) library construction methods and gene cloning. In next-generation sequencing methods, the ligation step is critical in adding sequencing platform-specific adapters to the DNA fragments that are to be sequenced. Said improvement is achieved by the addition of single- or double-stranded DNA-binding proteins in the ligation step.

### BACKGROUND OF THE INVENTION

Double-stranded nucleic acids containing blunt ends or cohesive (sticky) ends with an overhang of one or more nucleotides can be joined by means of intermolecular or intramolecular ligation reactions. Examples for the methods for ligating at a specific site are DNA ligation reactions of cohesive ends of DNA fragments, which have been cleaved by a restriction enzyme, or blunt-ends of DNA fragments. Such ligation reactions are commonly used in molecular biology applications, such as next-generation sequencing and gene cloning.

Next-generation sequencing (NGS), also known as high-throughput sequencing allows to acquire genome-wide data using highly parallel sequencing approaches for molecular biology applications, in vitro clinical diagnostics, or for forensics. Such applications include e.g. de novo genome sequencing, transcriptome sequencing and epigenomics, as well as genetic screening for the identification of rare genetic variants and for efficient detection of either inherited or somatic mutations in cancer genes.

Hence, several sequencing platforms have been developed, which allow for low-cost, high-throughput sequencing. Such platforms include Illumina® (Solexa) platforms, and Ion torrent Proton/PGM by Life Technologies/Thermo Fisher Scientific. NGS technologies, NGS platforms and common applications/fields for NGS technologies are e.g. reviewed in Voelkerding et al. (Clinical Chemistry 55:4 641-658, 2009), and Metzker (Nature Reviews/Genetics Volume 11, January 2010, pages 31-46).

Three main steps exist in NGS on most current platforms: preparation of the sample for high-throughput sequencing, immobilization on a suitable surface, and the actual sequencing. The preparation step involves random fragmentation of the genomic DNA and addition of adapter sequences to the fragment ends. The commonly used method to generate platform-specific NGS libraries uses multi-step enzymatic reaction protocols to ligate adapters to the DNA fragments to be analyzed.

First, DNA fragments are generated with mechanical, chemical, or enzymatic fragmentation or by target-specific PCR. Subsequently, the DNA fragments are end-repaired. The end-repair step requires at least two enzymes: (a) a polynucleotide kinase, normally the T4 Polynucleotide Kinase (PNK) that phosphorylates the 5′-terminus of the double stranded DNA fragments; and (b) an enzyme or enzymes with polymerase and exonuclease activities that make the ends of the DNA fragments blunt by either fill-in or trimming reactions, such as e.g. T4 DNA Polymerase. After the end-repair step, for sequencing on platforms, such as those provided by Illumina®, a so-called A-addition step is required, which generates a terminal adenine as a docking site for the sequencing adapters that have an overhang formed by thymidine nucleotides, i.e. a T-overhang. In this step, an A-overhang is added to the 3′-terminus of the end-repaired PCR product, e.g. by Klenow Fragment exo-, the large fragment of the DNA polymerase I having 5′→3′ polymerase activity, but lacking both 3′→5′ exonuclease activity and 5′→3′ exonuclease activity. Alternatively, the A-addition step can also be facilitated with enzymes having terminal nucleotide transferase activity, such as the Taq polymerase. Following the A-addition step, the sequencing adapter can be ligated to the DNA by a ligase, such as the T4 DNA Ligase. For other sequencing platforms, such as Ion Torrent PGM/Proton by Life Technologies®, the A-addition step is not required and blunt-ended adapters are ligated by a T4 DNA ligase directly to the end-repaired DNA fragments.

Currently, most available library construction methods can only reliably generate a sequencing library from more than 1 ng starting materials. The low efficiency of current library generation methods can be a draw-back if a sequencing library needs to be constructed from samples where only a small amount of input DNA is available, such as biopsy samples, circulating nucleic acids, ancient DNA, and FFPE samples.

Thus, there is a need in the art for sample preparation methods for a NGS library protocol generation and ligation preparation for gene cloning, especially when small amounts of DNA are to be analyzed.

### SUMMARY OF THE INVENTION

The present invention relates to single- or double-stranded DNA-binding proteins, which improve the efficiency and specificity of ligation reactions in molecular biology applications, such as gene library generation and gene cloning.

In particular, such an improvement is disclosed herein for gene library generation, where the presence of a single- or a double-stranded DNA binding protein leads to an enhanced library yield and libraries with a higher specificity. In the context of sequencing library construction, high ligation specificity means that only the end-repaired DNA fragments and adapters, not DNA fragments or adapters by themselves, are ligated together. This is necessary to prevent sequencing artefacts that can arise from DNA or adapter dimers and concatemers. Surprisingly, the addition of single- or double-stranded DNA-binding proteins can significantly enhance both the yield and the specificity of the ligation product, and hence both the yield and the specificity of a generated NGS library.

Similarly, in the presence of said proteins in gene cloning, the insert gene DNA and the vector DNA are ligated together, not the insert gene DNA and the vector DNA by themselves.

The yield of ligated dsDNA may increase by at least 3-fold in the presence of ss- or dsDNA binding proteins.

One aspect of the present invention refers to methods of generating a circular double-stranded DNA (dsDNA) or a sequencing library, wherein the method comprises circulating a dsDNA or ligating a first and a second dsDNA in the presence of a DNA ligase and a single-stranded DNA binding protein or a double-stranded DNA-binding protein.

In some embodiments, the method of generating a sequencing library comprises further steps, preceding the ligation, of:

(i) providing DNA fragments;
(ii) end-repairing the DNA fragments by a polynucleotide kinase enzyme and an enzyme with polymerase and exonuclease activities; and
(iii) optionally adding a terminal adenine to the end of the end-repaired DNA fragments by a deoxynucleotidyl transferase enzyme.

In some embodiments, said method further comprises the subsequent steps of purification and size-selection of the ligated fragments for sequencing.

In some embodiments, the adapter-ligated fragments are amplified prior to sequencing.

Another aspect of the invention refers to a kit comprising

(i) a DNA ligase; and
(ii) a single-stranded DNA (ssDNA) binding protein or a double-stranded DNA (dsDNA)-binding protein.

In some embodiments, the kit comprises:

(i) a polynucleotide kinase and an enzyme with polymerase and exonuclease activities;
(ii) optionally a deoxynucleotidyl transferase;
(iii) a DNA ligase;
(iv) a single-stranded or a double-stranded DNA binding protein; and
(v) optionally a reaction buffer.

In preferred embodiments, any of the kits comprises a mixture of a ligase, a single-stranded DNA (ssDNA) binding protein or a double-stranded DNA (dsDNA) binding protein, and optionally a reaction buffer.

In a preferred embodiment, the enzyme with polymerase and exonuclease activities is a DNA polymerase.

In the methods or kits referenced above, the polynucleotide kinase enzyme is the T4 Polynucleotide Kinase (PNK), the enzyme with polymerase and exonuclease activities is T4 DNA Polymerase, and/or the deoxynucleotidyl transferase enzyme is a Taq polymerase or a Klenow Fragment exo-.

In some embodiments, the present invention refers to ligation methods, wherein both the first and the second dsDNAs comprise two ssDNA ends, whereby each of the ssDNA ends of the first dsDNA ligates with each of the complementary ss ends of the second dsDNA to provide ligated circular dsDNA. Herein it is preferred that the first or the second DNA is capable of conferring the ability to auto-replicate within competent cells.

In the methods or kits referenced above, the DNA binding protein is a viral, bacterial, archaeal, or eukaryotic single-stranded DNA binding protein or double-stranded DNA binding protein.

In some embodiments, the DNA ligase in any of the above methods or kits is a T3 DNA ligase or a T4 DNA ligase. In other embodiments, the ligase is a T7 DNA ligase or an Ampligase®.

In some embodiments of the above methods, each of the first and the second dsDNA have one or two single stranded DNA (ssDNA) end(s). This/these ssDNA end(s) is/are less than 20 nucleotides (nt) in length.

### BRIEF DESCRIPTION OF THE DRAWINGS

FIG. 1: The Agilent Bioanalyzer graph shows the size distribution and quantity of the sequencing libraries generated with either standard ligation condition (‘Control’, blue line), or additional ET SSB (Extreme Thermostable Single-Stranded DNA Binding Protein) in the Ligation reaction (‘ET SSB in Ligation’, red line). As shown in FIG. 1, the addition of ET SSB in the ligation reaction can significantly improve the yield of the library, as determined by the increased peak heights at about 500 bp; as well as the specificity, as determined by the decreased peak heights at about 130 bp, which represent an adapter-dimer.

FIG. 2: The diagram shows the qPCR quantification results of the concentrations of the sequencing libraries generated with either standard ligation condition (‘Control’, blue line), or additional ET SSB in the Ligation reaction (‘ET SSB in Ligation’, red line).

### DETAILED DESCRIPTION OF THE INVENTION

### Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry).

In practicing the present invention, many conventional techniques in molecular biology, microbiology, and recombinant DNA may be used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods In Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

The term “SSB” refers to a single-stranded DNA binding protein.

The term “dsDNA” refers to a double-stranded DNA binding protein.

The term “ET SSB” (Extreme Thermostable Single-Stranded DNA Binding Protein) is a single-stranded DNA binding protein isolated from a non-thermophilic organism or a thermophilic microorganism.

The term “thermophilic organism” is an organism that lives in hot environments (e.g. hot springs) with temperatures around the boiling point of water. Thermophilic organisms include, but they are not restricted to bacteria and archaea, such as genera of Pyrococcus, Thermococcus. Palaeococcus, Acidianus, Pyrobaculum, Pyrodictium, Pyrolobus, Methanopyrus, Methanothermus, thermophilic Methanococci like Mc. jannaschii, Fervidobacterium and Thermotoga, and aerobic thermophilic organisms selected from the genera of Thermus, Bacillus, Deinococcus, Thermoactinomyces, as well as the species Aeropyrum pernix, Metallosphaera sedula and other Metallosphaera species, Sulfolobus solfataricus, Sulfolobus tokodaii, Thermoplasma acidophilum, and Thermoplasma volcanium.

The terms “next generation sequencing” and “high-throughput sequencing” are used as synonyms.

The term “library” refers to a large number of nucleic acid fragments, here the collection of DNA fragments for sequencing analysis. The libraries referred to herein are generated by fragmentation of a sample to be analyzed, end-repairing, optionally addition of a terminal adenine, and ligation of fragments into adapters. Optionally, the purified DNA fragments are amplified or enriched before they are sequenced.

The term “high ligation specificity” means that only the end-repaired DNA fragments and adapters, not DNA fragments or adapters by themselves, are ligated together. The specificity itself can be measured by methods known to the skilled person, such as PCR.

As used herein, the term “about” when used together with a numerical value (e.g., a pH value or a percentage value) is intended to encompass a deviation of 20%, preferably 10%, more preferably 5%, even more preferably of 2%, and most preferably of 1% from that value. When used together with a numerical value it is at the same time to be understood as individually disclosing that exact numerical value as a preferred embodiment in accordance with the present invention.

The terms “next generation sequencing” and “high-throughput sequencing” are used as synonyms.

The term “restriction endonuclease” is used herein in its commonly accepted sense as a site specific endodeoxyribonuclease and isoschizomers thereof. Restriction endonucleases are well-known compounds as is the method of their preparation; see for example Roberts, Critical Reviews in Biochemistry, November 1976, pages 123-164. Representative restriction endonucleases which may be employed in the method of the invention include, but are not restricted to: Alu I, Ava I, Ava II, Bal I, Bam HI, Bcl I, Bgl I, Bst E II, Eco R I, Hae II, Hae III, Hinc II, Hind II, Hind III, Hinf I, Hha I, Hpa I, Hpa II, Hph I, Hin 389I, Kpn II, Pst I, Rru I, Sau 3A, Sal I, Sma I, Sst I, Sst II, Tac I, Taq I, Xba I, Xho I and the like, many of which are commercially available (e.g. NEB, Promega, Life Technologies, and Thermo Scientific). Other restriction endonucleases which may be employed and their preparation are listed in e.g. Roberts, pages 127-130.

The term “median fragment size” means that half of the fragments have a longer length and half of the fragments have a shorter length.

As used herein, the term “comprising” is to be construed as encompassing both “including” and “consisting of”, both meanings being specifically intended, and hence individually disclosed embodiments in accordance with the present invention.

“nt” is an abbreviation of “nucleotides”.

“bp” is an abbreviation of “base pair”.

“T4 Polynucleotide Kinase” refers to an enzyme that catalyzes the transfer and exchange of Pᵢ from the γ position of ATP to the 5′-hydroxyl terminus of polynucleotides (double- and single-stranded DNA and RNA) and nucleoside 3′-monophosphates.

“T4 DNA Polymerase” refers to an enzyme that catalyzes the synthesis of DNA in the 5′→3′ direction and requires the presence of template and primer. This enzyme has a 3′→5′ exonuclease activity which is much more active than that found in DNA Polymerase I (E. coli). T4 DNA Polymerase does not exhibit 5′→3′ exonuclease activity.

“Klenow fragment exo-” or “Klenow fragment (3′→5′ exo-)” refers to an N-terminal truncation of DNA Polymerase I which retains polymerase activity, but has lost the 5′→3′ exonuclease activity and the 3′→5′ exonuclease activity.

“Taq polymerase” refers to a highly thermostable DNA polymerase from the thermophilic bacterium Thermus aquaticus. The enzyme catalyzes 5′→3′ synthesis of DNA, has no detectable 3′→5′ exonuclease (proofreading) activity and possesses low 5′→3′ exonuclease activity. In addition, Taq DNA Polymerase exhibits deoxynucleotidyl transferase activity, which is often applied in the addition of additional adenines at the 3′-end of PCR products to generate 3′ adenine overhangs.

The terms “deoxynucleotidyl transfer”, “terminal nucleotide addition” and “terminal nucleotide transfer” are used herein as synonyms.

“T3 DNA ligase” refers to an ATP-dependent dsDNA ligase from bacteriophage T3. It catalyzes the formation of a phosphodiester bond between adjacent 5′ phosphate and 3′ hydroxyl groups of duplex DNA. The enzyme joins both cohesive (sticky) and blunt ends.

“T4 DNA Ligase” refers to an enzyme that catalyzes the formation of a phosphodiester bond between juxtaposed 5′ phosphate and 3′ hydroxyl termini in double-stranded DNA or RNA. This enzyme joins both blunt end and cohesive (sticky) ends.

“T7 DNA Ligase” is an ATP-dependent ligase from bacteriophage T7. This enzyme joins cohesive (sticky) ends and it is suitable for nick sealing. Blunt-end ligation does not occur in the presence of a T7 ligase.

“Ampligase®” refers to a DNA Ligase that catalyzes NAD-dependent ligation of adjacent 3′-hydroxylated and 5′-phosphorylated termini in duplex DNA structures that are stable at high temperatures. The enzyme that catalyzes the formation of a phosphodiester bond between juxtaposed 5′ phosphate and 3′ hydroxyl termini in double-stranded DNA or RNA. This enzyme joins cohesive (sticky) ends. The half-life of Ampligase® is 48 hours at 65° C. and more than 1 hour at 95° C. In most cases, the upper limit on reaction temperatures with Ampligase® is determined by the Tm of the DNA substrate. Under conditions of maximal hybridization stringency, nonspecific ligation is nearly eliminated.

The term “reaction buffer” refers to a conventional buffer for DNA ligation known to the skilled person. The reaction buffer can comprise, for example, 50 mM Tris-HCl, 10 mM MgCl₂, 1 mM ATP, 10 mM DTT, and a pH of 7.5 at 25° C.

The term “melting temperature of double-stranded nucleic acids” is the temperature, at which half of the DNA strands are in the random coil or single-stranded (ssDNA) state, and half of the DNA strands are in a double-stranded state. Tm depends on the length of the DNA molecule and its specific nucleotide sequence, in particular, the guanine (G) and cytosine (C) content. In this context, the double-stranded nucleic acids refer to dsDNA, dsRNA or RNA:DNA hybrids. The melting temperature also depends on the ionic strength of the solution. One may calculate the melting temperature Tm of any given DNA hybrid as shown:

Tm=81.5° C.+0.41(% G+% C)−550/n
n=probe length (number of nucleotides).

The equation for calculating the melting temperature used above refers to the melting temperature that was measured under standard conditions (about 0.8 M NaCl, neutral pH (about pH 7.0)). The melting temperature can be measured experimentally by assessing dissociation-characteristics of double-stranded DNA during heating, which is visualized by UV spectroscopy, or by fluorescence measurements, where a fluorescent dye is used for readout, such as SYBR® Green I, YO-PRO-I®, or ethidium bromide.

The term “high stringency” refers to conditions, under which ability of nucleic acids with certain mismatched bases to hybridize is reduced or completely eliminated. Higher stringency conditions result in a higher ratio of the amount of hybridization of sequences with no mismatches when compared to the amount of hybridization of sequences with one or more mismatches.

The term “PCR” refers to polymerase chain reaction, which is a standard method in molecular biology for DNA amplification.

The term “qPCR” refers to quantitative real-time PCR, a method used to amplify and simultaneously detect the amount of amplified target DNA molecule fragments. The process involves PCR to amplify one or more specific sequences in a DNA sample. At the same time, a detectable probe, typically a fluorescent probe, is included in the reaction mixture to provide real-time quantification. Two commonly used fluorescent probes for quantification of real-time PCR products are: (1) non-sequence-specific fluorescent dyes (e.g., SYBR® Green) that intercalate into double-stranded DNA molecules in a sequence non-specific manner, and (2) sequence-specific DNA probes (e.g., oligonucleotides labeled with fluorescent reporters) that permit detection only after hybridization with the DNA targets or after incorporation into PCR products.

The term “DNA” in the present invention relates to any one of viral DNA, prokaryotic DNA, archaeal DNA, and eukaryotic DNA. The DNA may also be obtained from any one of viral RNA, and mRNA from prokaryotes, archaea, and eukaryotes by generating complementary DNA (cDNA) by using a reverse transcriptase.

The term “transcription factor” refers to modular proteins that affect regulation of gene expression include, but are not restricted to AAF, ab1, ADA2, ADA-NF1, AF-1, AFP1, AhR, AIIN3, ALL-1, alpha-CBF, alpha-CP1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arnt, Arnt (774 M form), ARP-1, ATBF1-A, ATBF1-B, ATF, ATF-1, ATF-2, ATF-3, ATF-3deltaZIP, ATF-a, ATF-adelta, ATPF1, Barhl1, Barhl2, Barx1, Barx2, Bcl-3, BCL-6, BD73, beta-catenin, Bin1, B-Myb, BP1, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, Chx10, CLIM1, CLIM2, CNBP, CoS, COUP, CP1, CP1A, CP1C, CP2, CPBP, CPE binding protein, CREB, CREB-2, CRE-BP1, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin T1, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, Dlx4 (long isoform), Dlx-4 (short isoform, Dlx-5, Dlx-6, DP-1, DP-2, DSIF, DSIF-p14, DSIF-p160, DTF, DUX1, DUX2, DUX3, DUX4, E, E12, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EllaE-A, EllaE-B, EllaE-Calpha, EllaE-Cbeta, EivF, Elf-1, Elk-1, Emx-1, Emx-2, Emx-2, En-1, En-2, ENH-bind. prot., ENKTF-1, EPAS1, epsilonF1, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRLIP2, Fli-1, Fos, FOXB1, FOXC1, FOXC2, FOXD1, FOXD2, FOXD3, FOXD4, FOXE1, FOXE3, FOXF1, FOXF2, FOXG1a, FOXG1b, FOXG1c, FOXH1, FOXI1, FOXJ1a, FOXJ1b, FOXJ2 (long isoform), FOXJ2 (short isoform), FOXJ3, FOXK1a, FOXK1b, FOXK1c, FOXL1, FOXM1a, FOXM1b, FOXM1c, FOXN1, FOXN2, FOXN3, FOXO1a, FOXO1b, FOXO2, FOXO3a, FOXO3b, FOXO4, FOXP1, FOXP3, Fra-1, Fra-2, FTF, FTS, G factor, G6 factor, GABP, GABP-alpha, GABP-beta1, GABP-beta2, GADD 153, GAF, gammaCMT, gammaCAC1, gammaCAC2, GATA-1, GATA-2, GATA-3, GATA-4, GATA-5, GATA-6, Gbx-1, Gbx-2, GCF, GCMa, GCNS, GF1, GLI, GLI3, GR alpha, GR beta, GRF-1, Gsc, Gscl, GT-IC, GT-IIA, GT-IIBalpha, GT-IIBbeta, H1TF1, HITF2, H2RIIBP, H4TF-1, H4TF-2, HAND1, HAND2, HB9, HDAC1, HDAC2, HDAC3, hDaxx, heat-induced factor, HEB, HEB1-p67, HEB1-p94, HEF-1 B, HEF-1T, HEF-4C, HEN1, HEN2, Hesxl, Hex, HIF-1, HIF-1alpha, HIF-1beta, HiNF-A, HiNF-B, HINF-C, HINF-D, HiNF-D3, HiNF-E, HiNF-P, HIP1, HIV-EP2, HIf, HLTF, HLTF (Met123), HLX, HMBP, HMG I, HMG 1(Y), HMG Y, HMGI-C, HNF-1A, HNF-1B, HNF-1C, HNF-3, HNF-3alpha, HNF-3beta, HNF-3gamma, HNF4, HNF-4alpha, HNF4alpha1, HNF-4alpha2, HNF-4alpha3, HNF-4alpha4, HNF4gamma, HNF-6alpha, hnRNP K, HOX11, HOXA1, HOXA10, HOXA10 PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXB5, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Id1, Id1 H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, iriB, IRX2a, Irx-3, Irx-4, ISGF-1, ISGF-3, ISGF3alpha, ISGF-3gamma, Ist-1, ITF, ITF-1, ITF-2, JRF, Jun, JunB, JunD, kappay factor, KBP-1, KER1, KER-1, Kox1, KRF-1, Ku autoantigen, KUP, LBP-1, LBP-1a, LBX1, LCR-F1, LEF-1, LEF-1B, LF-A1, LHX1, LHX2, LHX3a, LHX3b, LHXS, LHX6.1a, LHX6.1b, LIT-1, Lmo1, Lmo2, LMX1A, LMX1B, L-My1 (long form), L-My1 (short form), L-My2, LSF, LXRalpha, LyF-1, LyI-1, M factor, Mad1, MASH-1, Max1, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2D0B, MEF-2DA0, MEF-2DA′0, MEF-2DAB, MEF-2DA′B, Meis-1, Meis-2a, Meis-2b, Meis-2c, Meis-2d, Meis-2e, Meis3, Meox1, Meox1a, Meox2, MHox (K-2), Mi, MIF-1, Miz-1, MM-1, MOP3, MR, Msx-1, Msx-2, MTB-Zf, MTF-1, mtTF1, Mxi1, Myb, Myc, Myc 1, Myf-3, Myf-4, Myf-5, Myf-6, MyoD, MZF-1, NC1, NC2, NCX, NELF, NER1, Net, NF III-a, NF NF NF-1, NF-1A, NF-1B, NF-1X, NF-4FA, NF-4FB, NF-4FC, NF-A, NF-AB, NFAT-1, NF-AT3, NF-Atc, NF-Atp, NF-Atx, NfbetaA, NF-CLE0a, NF-CLE0b, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaB1, NF-kappaB1, precursor, NF-kappaB2, NF-kappaB2 (p49), NF-kappaB2 precursor, NF-kappaE1, NF-kappaE2, NF-kappaE3, NF-MHCIIA, NF-MHCIIB, NF-muE1, NF-muE2, NF-muE3, NF-S, NF-X, NF-X1, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A v1, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-Sb, NP-TCII, NR2E3, NR4A2, Nrf1, Nrf-1, Nrf2, NRF-2beta1, NRF-2gamma1, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otx1, Otx2, OZF, p107, p130, p28 modulator, p300, p38erg, p45, p49erg, -p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-1a, Pbx-1b, Pbx-2, Pbx-3a, Pbx-3b, PC2, PC4, PC5, PEA3, PEBP2alpha, PEBP2beta, Pit-1, PITX1, PITX2, PITX3, PKNOX1, PLZF, PO-B, Pontin52, PPARalpha, PPARbeta, PPARgamma1, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (BJA-B), PU.1, PuF, Pur factor, R1, R2, RAR-alpha1, RAR-beta, RAR-beta2, RAR-gamma, RAR-gamma1, RBP60, RBP-Jkappa, Rel, RelA, RelB, RFX, RFX1, RFX2, RFX3, RFXS, RF-Y, RORalpha1, RORalpha2, RORalpha3, RORbeta, RORgamma, Rox, RPF1, RPGalpha, RREB-1, RSRFC4, RSRFC9, RVF, RXR-alpha, RXR-beta, SAP-1a, SAP1b, SF-1, SHOX2a, SHOX2b, SHOXa, SHOXb, SHP, SIII-p110, SIII-p15, SIII-p18, SIM′, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Sp1, Sp2, Sp3, Sp4, Sph factor, Spi-B, SPIN, SRCAP, SREBP-1a, SREBP-1b, SREBP-1c, SREBP-2, SRE-ZBP, SRF, SRY, SRP1, Staf-50, STAT1alpha, STAT1beta, STAT2, STAT3, STAT4, STATE, T3R, T3R-alpha1, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-1beta, Tal-2, TAR factor, TBP, TBX1A, TBX1B, TBX2, TBX4, TBXS (long isoform), TBXS (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbeta1, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-MO15, TFIIH-p34, TFIIH-p44, TFIIH-p62, TFIIH-p80, TFIIH-p90, TFII-1, Tf-LF1, Tf-LF2, TGIF, TGIF2, TGT3, THRA1, TIF2, TLE1, TLX3, TMF, TR2, TR2-11, TR2-9, TR3, TR4, TRAP, TREB-1, TREB-2, TREB-3, TREF1, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-1A, vHNF-1B, vHNF-1C, VITF, WSTF, WT1, WT1I, WT1 I-KTS, WT1 I-de12, WT1-KTS, WT1-de12, X2BP, XBP-1, XW-V, XX, YAF2, YB-1, YEBP, YY1, ZEB, ZF1, ZF2, ZFX, ZHX1, ZIC2, ZID, and ZNF174.

### Single-Stranded and Double-Stranded DNA-Binding Proteins

### Single-Stranded DNA-Binding Proteins

Single stranded (ss) DNA-binding proteins (SSBs) are essential to virtually all aspects of DNA metabolism. These proteins, exemplified by the Escherichia coli ssDNA-binding protein (SSB) in bacteria (Sancar, A., et al., Proc. Natl. Acad. Sci. USA 78, 4274-4278 (1981), Lohman, T. M. et al., Annu. Rev. Biochem. 63, 527-570 (1994)) and the human replication protein-A (RPA) complex in eukarya (Fairman, M. P. et al., EMBO J. 7, 1211-1218 (1988); Wold, M. S. et al., Proc. Natl. Acad. Sci. USA 85, 2523-2527 (1988); and Wold, M. S., Annu. Rev. Biochem. 66, 61-92 (1997)), are required for in vitro DNA replication, and they are key components in DNA recombination and repair. SSB may be prokaryotic, eukaryotic, archaeal, or viral.

In some embodiments, SSB may be prokaryotic, preferably bacterial. In other embodiments, SSB may be archaeal. In yet other embodiments, SSB may be eukaryotic. In still other embodiments, SSB may be viral.

Prokaryotic SSB may be bacterial. Examples of bacterial SSB include, but are not restricted to those from Escherichia coli (E. coli) (E. coli SSB), E. coli RecA, Salmonella typhimurium, Bacillus licheniformis, Campylobacter jejuni, Pseudomonas syringae and Listeria innocua, as well as Thermus aquaticus, Thermus thermophiles, M. smegmatis, and D. radiodurans SSB.

Replication protein A (RPA) is a eukaryotic SSB. It is a heterotrimeric single-stranded DNA-binding protein that is highly conserved in eukaryotes.

Accordingly, envisaged are proteins, in particular, single-stranded DNA binding proteins isolated from the above organisms, or which are recombinantly expressed, but comprise the amino acid sequence of the single-stranded DNA binding proteins of the above organisms, and which maintain their activities and are stable at high temperatures. The amino acid sequence of said proteins may be identical. Alternatively, the sequence identity may be at least 90%, 95%, 96%, 97%, 98%, or 99% identical. Proteins may originate and be isolated from non-thermophilic bacteria, such as E. coli and B. subtilis. Alternatively, the proteins may originate and be isolated from thermophilic bacteria or archaea (described in more detail below). The aforementioned proteins may alternatively be recombinantly expressed proteins having an identical sequence to that of the isolated proteins. A characteristic feature of these thermophilic proteins is that they survive a heating step of about 65° C. to about 100° C. (most preferably about 80° C. to about 95° C.), for a sufficient period of time (e.g. at least about 1-3 minutes, and preferably for at least 5 minutes).

Although functionally equivalent, the prokaryotic SSB protein and the eukaryotic counterpart, Replication Protein A (RPA) have very different protein structures. Bacterial SSB proteins are encoded by a single gene, although the active form is a homotetramer of SSB where each monomer contributes one ssDNA-binding domain, whereas the eukaryotic counterpart is a heterotrimer.

A bacterial SSB monomer has two distinct domains: (i) a conserved N-terminal domain responsible for (homo)tetramerization and DNA-binding, and (ii) a less conserved C-terminal domain important for the interaction of SSBs with various proteins. Many bacteria encode two SSBs that differ in size. For example, in B. subtilis, it was shown that the larger SSB is an essential protein and participates in DNA replication, while the short SSB, lacking most of the C-terminal domain, is non-essential but plays a role in natural transformation. The SSBs referred to herein refer to any bacterial SSB, which comprises a fully functional conserved N-terminal domain. Depending on the salt concentration in vitro, about 35 nucleotides bind to only two of the SSB subunits (low salt concentrations), or about 65 nucleotides of DNA wrap around the SSB tetramer and contact all four of its subunits (high salt concentrations) (Bujalowski and Lohman, Biochemistry, 1986, 25, 7799-7802). About 22-50 nucleotides are required for an E. coli SSB and homologues thereof to efficiently interact with ssDNA (http://www.bioptixinc.com/applications/ssb/).

The eukaryotic RPA complex is composed of three distinct subunits (heterotrimer), which are referred to as RPA70, RPA32 and RPA14. In DNA-processing events, RPA also interacts with many additional nuclear proteins. This interaction both regulates, and is regulated by, an interaction with ssDNA. The major ssDNA-binding activity of RPA is located in the central part of the RPA70 subunit (amino acids (aa) 181-422; RPA70₁₈₁₋₄₂₂ of human RPA and corresponding counterparts in other eukarya). Structural analysis of this fragment in complex with a (dC)₈-oligonucleotide revealed two structurally similar copies of a structural domain known as an OB (oligonucleotide/oligosaccharide binding)-fold. The two DNA-binding domains (DBDs) of RPA70, DBD-A (aa 181-290) and DBD-B (300-422), contact ssDNA in tandem. Each domain directly contacts 3 nt, with 2 nt filling the space between domains (Bochareva et al., EMBO J., 2001).

Archaeal ssDNA-binding proteins include, but are not restricted to SSB from Methanococcus jannaschii, Methanobacter theromoautotrophicum, Archaeoglobus fulgidus, Sulfolobus Solfataricus P2 (SSOB), and Thermococcus kodakarensis.

The viral single-stranded DNA-binding proteins include, but are not restricted to viral SSB, such as adenovirus-encoded DNA binding protein, EBV BALF2 protein, Herpes simplex virus type 1 single-strand DNA binding protein ICP8, T4 gene 32 protein (T4 gp32), T4 gene 44/62 protein, T7 SSB, coliphage N4 SSB, adenovirus DNA binding protein (Ad DBP or Ad SSB), and calf thymus unwinding protein (UP1). Chase et al., Ann. Rev. Biochem. 55:103-36 (1986); Coleman et al., CRC Critical Reviews in Biochemistry 7(3):247-289 (1980); Lindberg et al., J. Biol. Chem. 264:12700-08 (1989); and Nakashima et al., FEBS Lett. 43: 125 (1974).

### Double-Stranded DNA-Binding Proteins

DNA-binding proteins include, but are not restricted to transcription factors which modulate the process of transcription, histone proteins, as well as antibodies, which have been designed to attach to dsDNA. These proteins comprise domains including, but not restricted to the zinc finger, ring finger, the helix-turn-helix, and the leucine zipper motif that facilitate binding to nucleic acid. Transcription factors modulate gene expression, replication, and recombination and are involved in many biological processes, such as cell growth and differentiation. In preferred embodiments, the transcription factor is non-sequence specific.

A further DNA-binding protein is the bacterial histone-like nucleoid-structuring (H-NS) protein. In eukaryotes, histone proteins comprise the proteins H1/H5, H2A, H2B, H3, and H4.

Single-stranded and double-stranded DNA binding proteins referred to above may be obtained by recombinant expression in a suitable expression host, such as E. coli, Pichia pastoris, Spodoptera frugiperda, or mammalian expression host cells, such as HEK or CHO cells. Alternatively, said proteins may be isolated from prokaryotic, eukaryotic or archaeal cells expressing them endogenously.

In preferred embodiments, the methods or kits of this invention refer to double- or single-stranded DNA binding protein or homologues thereof, wherein a homologue shares a protein sequence identity to the above mentioned of at least 50%, preferably, at least 60%, and more preferably at least 90%, 95%, 96%, 97%, 98%, or 99%.

### Methods

The present invention refers to ligation methods, in particular to gene cloning methods and methods of generating sequencing libraries.

In particular, the method referred herein is characterized in that the ligation step efficiency and specificity is increased by applying an SSB or a double-stranded DNA binding protein to a ligation reaction, which is a critical step in gene cloning and in next generation sequencing library generation.

### Next Generation Sequencing

One aspect of the present invention refers to a method of generating a sequencing library, wherein the method comprises ligating a first and a second dsDNA in the presence of a DNA ligase and a single-stranded DNA binding protein or a double-stranded DNA-binding protein.

In some embodiments, the method of generating a sequencing library comprises further steps, preceding the ligation, of:

(i) providing DNA fragments;
(ii) end-repairing the DNA fragments by a polynucleotide kinase enzyme and an enzyme with polymerase and exonuclease activities, preferably DNA polymerase; and
(iii) optionally adding a terminal adenine to the end of the end-repaired DNA fragments by a deoxynucleotidyl transferase enzyme.

In preferred embodiments, the ligation is carried out in the presence of a single-stranded DNA-binding protein.

In preferred embodiments, the ligation is carried out under high stringency conditions.

In some embodiments, said method further comprises the subsequent steps of purification and size-selection of the ligated fragments for sequencing. In some embodiments, the adapter-ligated fragments are amplified prior to sequencing. The library fragments are subsequently sequenced by using sequencing platforms known to the person skilled in the art, such as Illumina® (Solexa) and Ion torrent Proton/PGM by Life Technologies/Thermo Fisher Scientific and other suitable high-throughput sequencing platforms.

The size of the DNA fragment length is a key factor for library construction and for sequencing. Typical median lengths of DNA fragments for NGS libraries are between about 150 bps and about 1000 bps, preferably between about 150 bps and about 600 bps, more preferably between about 200 bps and about 500 bps. Most preferably, the median length is about 200 bps, about 300 bps, or about 500 bps.

The preferred amount of DNA starting material for generating a NGS sequencing library and for subsequent sequence analysis ranges from about 1 pg to about 1 μg, preferably from about 10 pg to about 1 μg, and more preferably about 10 pg to about 1 ng. For genomic DNA analysis, the amount of starting material is preferably about 1 pg to about 1 μg, preferably from about 10 pg to about 1 μg, and more preferably about 10 pg to about 1 ng.

In some embodiments, the fragmentation step is mechanical. Preferably, the mechanical fragmentation is among others achieved by ultrasonic acoustic shearing, nebulization forces, sonication, hydrodynamic shearing (e.g. in French pressure cells or by needle shearing). More preferably, specific median fragment length sizes of DNA can be prepared e.g. by ultrasonic acoustic shearing, such as Adaptive Focused Acoustics (AFA)™ by using a Covaris® instrument, according to the manufacturer's instructions.

In some embodiments, the fragmentation of DNA step is chemical. Chemical shear may also be employed for the breakup of long RNA fragments. This is typically performed through heat digestion of RNA with a divalent metal cation (magnesium or zinc). The length of the RNA (115 bp-350 bp) can be adjusted by increasing or decreasing the time of incubation.

In some embodiments, the fragmentation step is enzymatic. Preferably, said enzymatic fragmentation is achieved by digestion of DNA by an endonuclease. Such endonucleases are described in more detail in the Definitions section. Preferably, the fragmentation may also be carried out by employing a transposase known to the person skilled in the art. When applying enzymes for the fragmentation reaction, said fragmentation step may be inactivated by heat.

Step (ii), the end-repair step, is carried out by an enzyme or two enzymes with (a) polynucleotide kinase activity (PNK) and (b) an enzyme with polymerase and exonuclease activities, whereby the exonuclease activity makes the ends of the DNA blunt by fill-in or trimming reactions. Preferably, the enzymes of step (ii) are a T4 Polynucleotide Kinase (PNK) and a T4 DNA Polymerase.

Step (iii), the A-addition step, is carried out by an enzyme, which generates an adenine docking site for adapters that have a thymidine overhang (T-overhang). Preferably, the enzyme of step (iii) is a Taq polymerase or Klenow Fragment exo-, the large fragment of the DNA polymerase I having 5′→3′ polymerase activity but lacking both 3′→5′ exonuclease activity and 5′→3′ exonuclease activity. In some preferred embodiments, the enzyme of step (iii) is a thermostable polymerase, preferably a Taq polymerase.

Step (iv), the ligation step, joins either blunt or cohesive (sticky) ends of DNA fragments with either blunt or cohesive (sticky) ends of adapter molecules. Successful ligation of cohesive (sticky) ends requires complementary sequences. In preferred embodiments, a fragment comprising terminal, i.e. 3′ adenine overhangs serves as a docking site for the sequencing adapters, which comprise a complementary terminal, i.e. 3′ thymidine overhang. By using such TA cloning it is not necessary to design a specific pair of primers for each DNA fragment to be analyzed. The same primers can be used for amplification of different templates provided that each template is modified by addition of the same universal primer-binding sequences to its 5′ and 3′ ends. The adapter sequence can therefore be any DNA fragment of interest, as long as it has a 3′ thymidine overhang.

Preferably, the ligation enzyme referred to above, in particular the enzyme of step (iv) is a T3 DNA ligase, T4 DNA ligase, T7 DNA ligase, an Ampligase®, or an E. coli DNA-ligase, whereby the T7 DNA ligase, the Ampligase® and the E. coli DNA-ligase only ligate cohesive (sticky) DNA.

In embodiments, where cohesive (sticky) end ligation, such as AT-ligation is envisioned, it is preferable to use T7 DNA ligase or an Ampligase®. For cohesive (sticky) end ligation under high stringency conditions Ampligase® is preferred, as its exceptional thermostability reduces the hybridization of mismatched base pairs. Ampligase® is the preferred ligase when thermophile ssDNA-binding or dsDNA-binding proteins, preferably thermophile ssDNA binding proteins are envisioned. Preferably, step (iv) comprises T4 DNA ligase when blunt ends are to be ligated.

The efficiency of the ligation step regarding specificity and library yield is improved by the addition of a SSB or dsDNA binding protein, which may be a prokaryotic, eukaryotic, archaeal, or viral protein.

In preferred embodiments, the ligation is carried out in the presence of a single-stranded DNA-binding protein. In some embodiments, the single-stranded DNA binding protein is eukaryotic, such as RPA. Alternatively, the eukaryotic single-stranded DNA binding protein is an antibody, binding to DNA with high affinity and specificity, which has been generated by the methods known to the skilled person.

In other preferred embodiments, the single-stranded DNA binding protein is prokaryotic, preferably bacterial. In more preferred embodiments under high stringency conditions, said bacterial protein is thermophile.

In yet other embodiments, the single-stranded DNA binding protein is archaeal. In preferred embodiments, under high stringency conditions, said archaeal protein is thermophile.

In preferred embodiments, the concentration of the single-stranded protein is about 2-10 ng/μL, more preferably, about 4-8 ng/μL, even more preferably about 5-7 ng/μL, or most preferably it is about 5.6 ng/μL.

The ligation step is carried out at 4-50° C., depending on the optimal temperature for the ligase's activity. For T3 DNA ligase, T4 DNA ligase, T7 DNA ligase, and E. coli DNA ligase, the preferred ligase temperature is 4-25° C. In embodiments, where Ampligase® is used, the ligation temperature is adapted according to the Tm of the DNA substrate to be ligated. Ampligase® is preferably used in combination with a thermophilic ssDNA-binding protein or a thermophilic dsDNA binding protein.

After the generation of the ligated fragments, said fragments are purified and size-selected on e.g. silicon containing surface of a binding matrix in the presence of a salt, preferably a chaotropic salt. The size of DNA molecules that bind to the binding matrix can be controlled e.g. by the salt concentration or the pH value of the binding mixture. Such purification is e.g. described in WO 2014/122288 A1. Suitable columns applying such a size selection method include the GeneRead™ Size Selection Kit. A further DNA size selection method includes agarose gel electrophoresis. The purified fragments may be used directly for subsequent sequencing. Alternatively, prior to the sequencing step, the purified fragments may be amplified for library enrichment by PCR-based methods known to the person skilled in the art, or by capture-by-hybridization, i.e. on-array or in-solution hybrid capture; or by capture-by-circularization, i.e. molecular inversion probe-based methods. Preferably, library enrichment is carried out by PCR amplification.

In some embodiments, the length of the nucleotide sequences of the ssDNA ends of dsDNA for the ligation methods in gene library generation referred to above is less than 20 nt or less than 12 nt, preferably the sequence length is less than 10 nt or less than 8 nt, more preferably 1-6 nt or 1-5 nt. In some embodiments the ssDNA length is 1 nt.

In embodiments, where the ssDNA region is 1 nt, ssDNA region of one DNA comprises a terminal (3′) adenine (A) and a the complementary ssDNA of the other DNA comprises a terminal (5′) thymidine (T). Alternatively the terminal ssDNA regions are (3′) cytosine (C) and the complementary terminal (5′) guanine (G).

In the most preferred embodiments, the ssDNA region of one DNA is terminal (3′) adenine (A) and the complementary ssDNA region of another dsDNA is a terminal (5′) thymidine (T).

In some embodiments, the ligation reaction in gene library generation is characterized in that the first dsDNA used in such ligation reactions comprises ssDNA regions at both of its termini, which may or may not be identical. Preferably, such terminal ssDNA regions are identical. More preferably, each of the terminal ssDNA regions comprises a terminal adenine. Each of the termini hybridizes under high stringency conditions with a complementary ssDNA region of a second dsDNA, respectively. Preferably, such a second dsDNA is a sequence adaptor. More preferably, such a sequence adaptor comprises a terminal thymidine.

### Gene Cloning

One aspect of the present invention refers to methods of generating circular dsDNA, wherein the method comprises ligating a first and a second dsDNA in the presence of a DNA ligase and a single-stranded DNA binding protein or a double-stranded DNA-binding protein.

In some embodiments, each of the first and the second dsDNA comprises two ssDNA regions, whereby the two ssDNA regions in one dsDNA may be identical or non-identical. The terminal ssDNA regions of the first and the second dsDNA hybridize under high stringency conditions in the presence of an SSB or a double-stranded DNA binding protein. Preferably, the ssDNA ends of the dsDNA to be ligated are complementary. In more preferred embodiments, the ligation is carried out in the presence of a single-stranded DNA-binding protein.

In the above methods, each of the ssDNA ends of the first dsDNA ligates with each of the ss ends of the second dsDNA to provide ligated circular dsDNA in the presence of a ssDNA binding protein or dsDNA binding protein, preferably ssDNA binding protein.

In preferred embodiments, the first DNA or the second DNA is capable of conferring the ability to auto-replicate within competent cells. The use of ligating nucleic acids in the presence or ssDNA or dsDNA binding proteins results in an increased number of transformed host cells after transformation with the ligated molecules with chemically transformed host cells or with host cells transformed by electroporation. A ligation yield increase may also be assessed by methods known to the skilled person, such as agarose gel electrophoresis.

The nucleotide sequence length of the DNA for ligation reactions, in particular gene cloning, more particular in vitro gene cloning is not restricted, as long as it agrees with the objective of this invention and accomplishes the functional effects of the invention. The appropriate scope of the aforementioned length can be understood by a person skilled in the art in the field of molecular biology.

The ratio of DNAs to be ligated is not restricted, and may be any, as long as they are within a range that does not adversely affect the correct ligation of each end. In embodiments for cloning of a specific gene, it is preferable to use the DNA to be ligated in a concentration that is equimolar to the DNA comprising the whole or partial gene. Other ratios of a vector and a gene to be inserted are 1:2, 1:5, 1:10, and 1:20. More preferably, such a ratio is 1:5.

When ligating two dsDNA, such as vector DNA and insert gene DNA, the vector DNA is preferably a DNA that can be introduced into a suitable competent cell, wherein it can auto-replicate.

Such vectors are selected according to the competent cells into which the ligate is introduced. For example, for E. coli, the commercially available vectors or plasmids can be used. Such vectors include, but are not restricted to pBR322, pQE series (N-terminus vectors: pQE-9, pQE-30, pQE31, pQE-32, and pQE-40; C-terminus vectors: pQE16, pQE60, pQE-70 (Qiagen), and pUC series (for example, pUC18, pSP64, pGEM-3, pBluescript). When using yeast as said cells, such vectors include, but are not restricted to Yep24, Ylp5. When using Bacillus, such vectors include, but are not restricted to pHY300 and PLK. Insect cell expression vectors include, but are not restricted to Easy Xpress pIX3.0 and pIX 4.0 (Qiagen). Vectors for E. coli, insect cell, and mammalian cell expression include, but are not restricted to pQE Trisystem vectors (Qiagen).

Preferably, the DNA ligation enzyme referred to above is a T3 DNA ligase, T4 DNA ligase, T7 DNA ligase, an Ampligase®, or an E. coli DNA-ligase, whereby the T7 DNA ligase, the Ampligase® and the E. coli DNA-ligase only ligate cohesive (sticky) DNA. In embodiments, where cohesive (sticky) end ligation, such as AT-ligation is envisioned, it is preferable to use T7 DNA ligase or Ampligase®. For cohesive (sticky) end ligation under high stringency conditions Ampligase® is preferred, as its exceptional thermostability permits very high hybridization stringency and ligation specificity. Ampligase® is also the preferred ligase when thermophile ssDNA-binding or dsDNA-binding proteins, preferably thermophile ssDNA binding proteins are applied to the ligation reaction. T4 DNA ligase is preferred when blunt ends are to be ligated.

The SSB or dsDNA binding protein, preferably SSB, may be prokaryotic, eukaryotic, archaeal, or viral. In some embodiments, the single-stranded DNA binding protein is eukaryotic, such as RPA. Alternatively, the eukaryotic single-stranded DNA binding protein is an antibody, binding to DNA with high affinity and specificity, which has been generated by the methods known to the skilled person.

In preferred embodiments, the single-stranded DNA binding protein is prokaryotic, preferably bacterial. In more preferred embodiments, said bacterial protein is thermophile.

In yet other preferred embodiments, the single-stranded DNA binding protein is archeal. In more preferred embodiments, said archeal protein is thermophile.

In preferred embodiments, the concentration of the single-stranded protein is about 2-10 ng/μL, more preferably, about 4-8 ng/μL, even more preferably about 5-7 ng/μL, or most preferably it is about 5.6 ng/μL.

The ligation step is carried out at 4-50° C., depending on the optimal temperature for the ligase's activity. For T3 DNA ligase, T4 DNA ligase, T7 DNA ligase and E. coli DNA ligase, the preferred ligase temperature is 4-25° C. In embodiments, where Ampligase® is used, the ligation temperature is adapted according to the Tm of the DNA substrate to be ligated.

In some embodiments, the length of the nucleotide sequences of the ssDNA ends of dsDNA for the ligation methods in gene cloning referred to above is less than 20 nt or less than 12 nt, preferably the sequence length is less than 10 nt or less than 8 nt, more preferably 1-6 nt or 1-5 nt. In some embodiments the ssDNA length is 1 nt.

In embodiments, where the ssDNA region is 1 nt, ssDNA region of one DNA comprises a terminal (3′) adenine (A) and a the complementary ssDNA of the other DNA comprises a terminal (5′) thymidine (T). Alternatively the terminal ssDNA regions are (3′) cytosine (C) and the complementary terminal (5′) guanine (G).

In the most preferred embodiments, the ssDNA region of one DNA is terminal (3′) adenine (A) and the complementary ssDNA region of another dsDNA is a terminal (5′) thymidine (T).

In some embodiments, the ligation reaction in gene cloning is characterized in that the first dsDNA used in such ligation reactions comprises ssDNA regions at both of its termini, which may or may not be identical. Each of the termini hybridizes under high stringency conditions with a complementary ssDNA region of a second dsDNA, respectively. Preferably, such the first dsDNA is a gene insert and the second dsDNA is a sequence adaptor, or vice versa.

### Kits

### Next Generation Sequencing

Another aspect of the present invention refers to kits comprising:

(i) a DNA ligase; and
(ii) a single-stranded DNA (ssDNA) binding protein or a double-stranded DNA (dsDNA)-binding protein.

In preferred embodiments, the kits comprise a mixture of a ligase, a single-stranded DNA (ssDNA) binding protein or a double-stranded DNA (dsDNA) binding protein, and optionally a reaction buffer.

In preferred embodiments, the invention relates to a kit comprising:

(i) a polynucleotide kinase and an enzyme, with polymerase and exonuclease activities, preferably DNA polymerase;
(ii) optionally a deoxynucleotidyl transferase;
(iii) a DNA ligase; and
(iv) a single-stranded or a double-stranded DNA binding protein.

In more preferred embodiments, the polynucleotide kinase enzyme is the T4 Polynucleotide Kinase (PNK); the enzyme with polymerase and exonuclease activity is the T4 DNA Polymerase; and/or the deoxynucleotidyl transferase enzyme is a Taq polymerase or a Klenow Fragment exo-.

The DNA ligase is a T3 DNA ligase, T4 DNA ligase, T7 DNA ligase, an Ampligase®, or an E. coli DNA-ligase, whereby the T7 DNA ligase, Ampligase® and the E. coli DNA-ligase only ligate cohesive (sticky) DNA. Therefore, more preferably, step (iv) comprises T4 DNA ligase when blunt ends are to be ligated. For cohesive (sticky) end ligation in step (iv), T7 DNA ligase or Ampligase® is preferred. For cohesive (sticky) end ligation under high stringency conditions Ampligase® is preferred as its exceptional thermostability permits high hybridization stringency and ligation specificity. Ampligase® is preferably used in combination with a thermophilic ssDNA-binding protein or a thermophilic dsDNA binding protein.

In the kits referenced above, the single-stranded DNA binding protein can be a viral, bacterial, archaeal, or eukaryotic single-stranded or double-stranded DNA binding protein, preferably single-stranded DNA binding protein.

In preferred embodiments, the single-stranded or double-stranded DNA binding protein is bacterial or archaeal. In more preferred embodiments, the DNA-binding protein is a single-stranded DNA-binding protein. It may originate from a non-thermophile or a thermophile bacterium. In ligation reactions under high stringency conditions the protein originates from a thermophile bacterium. In other preferred embodiments, the single-stranded DNA-binding protein is selected from a non-thermophile or a thermophile archaeon. In ligation reactions under high stringency conditions the protein originates from a thermophile archaeon.

In preferred embodiments, the concentration of the single-stranded protein is about 2-10 ng/μL, more preferably, about 4-8 ng/μL, even more preferably about 5-7 ng/μL, or most preferably it is about 5.6 ng/μL.

### Gene Cloning

In other preferred embodiments, the invention relates to a kit comprising

(i) a DNA ligase; and
(ii) a single- or a double-stranded DNA binding protein.

In preferred embodiments, any of the kits comprises a mixture of a ligase, a single-stranded DNA (ssDNA) binding protein or a double-stranded DNA (dsDNA)-binding protein, and optionally a reaction buffer.

In the kits referenced above, the single-stranded DNA binding protein can be a viral, bacterial, archaeal, or eukaryotic single-stranded or double-stranded DNA binding protein, single-stranded DNA binding protein.

In preferred embodiments, the single-stranded or double-stranded DNA binding protein is bacterial or archaeal. In more preferred embodiments, the single-stranded DNA-binding protein is selected from a non-thermophile or a thermophile bacterium or archaeon.

The DNA ligase is a T3 DNA ligase, T4 DNA ligase, T7 DNA ligase, an Ampligase®, or an E. coli DNA-ligase, whereby the T7 DNA ligase, Ampligase® and the E. coli DNA-ligase only ligate cohesive (sticky) DNA. Therefore, more preferably, step (iv) comprises T4 DNA ligase when blunt ends are to be ligated. For cohesive (sticky) end ligation, Ampligase® is preferred, as its exceptional thermostability permits high hybridization stringency and ligation specificity. Ampligase® is preferably used in combination with a thermophilic ssDNA-binding protein or a thermophilic dsDNA binding protein.

In the gene cloning kits referenced above, the single-stranded DNA binding protein can be a viral, bacterial, archaeal, or eukaryotic single-stranded or double-stranded DNA binding protein.

In preferred embodiments, of the above referenced gene-cloning kits, the single-stranded or double-stranded DNA binding protein is bacterial or archaeal. In more preferred embodiments, the single-stranded DNA-binding protein is selected from a non-thermophile or a thermophile bacterium. In ligation reactions under high stringency conditions the protein originates from a thermophile bacterium. In other preferred embodiments, the single-stranded DNA-binding protein is selected from a non-thermophile or a thermophile archaeon. In ligation reactions under high stringency conditions the protein originates from a thermophile archaeon.

In preferred embodiments, the concentration of the single-stranded protein is about 2-10 ng/μL, more preferably, about 4-8 ng/μL, even more preferably about 5-7 ng/μL, or most preferably it is about 5.6 ng/μL.

### EXAMPLES

gDNA from E. coli DH10B is sheared to an average fragment size of 300 bp (Covaris S220 Focused-ultrasonicator, Covaris), and 10 pg of sheared DNA is used for each library construction test. GeneRead™ DNA Library Prep I Core Kit, GeneRead™ DNA I Amp Kit, GeneRead™ Adapter I Set 12-Plex (72), and GeneRead™ Size Selection Kit (all from QIAGEN) are used according to manufacturer's instructions with the following modifications:

0.5 U of the Taq polymerase (QIAGEN) and 0.5 mM of dATP (QIAGEN) are added to the end-repair reaction; the temperature profile for end-repair reaction is 30 minutes at 25° C., and 30 minutes at 72° C., where the 72° C. step was used to both inactivate end-repair enzymes and utilize the terminal transferase activity of the Taq enzyme to add an adenine to the 3′ of the DNA fragments. The separate A-addition step using Klenow fragment (3′→5′ exo-) is therefore removed from the protocol.

0.05 μM of sequencing adapter was used in the ligation steps.

Following the ligation step, the library was first purified with the GeneRead Size Selection Kit (QIAGEN), then amplified for 22 cycles with adapter-specific primers in PCR (GeneRead DNA I Amp Kit, QIAGEN), and purified again with GeneRead Size Selection Kit (QIAGEN). The final sequencing libraries were qualified with Agilent Bioanalyzer High Sensitivity DNA Analysis Kit (Agilent) and quantified with the qPCR method (QuantiFast Sybr Green Kit, QIAGEN).

As a proof of principle, we used either standard ligation condition as described in manufacturer's instruction, or added 1 μl of ET SSB (Extreme Thermostable Single-Stranded DNA Binding Protein, 5.6 ng/μL, New England Biolabs) into the ligation reaction.

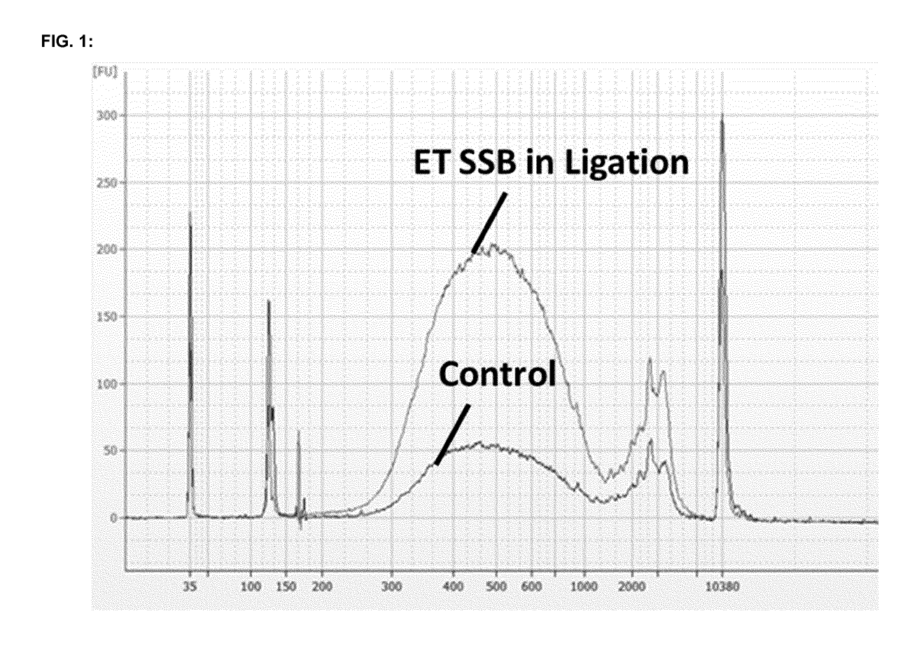

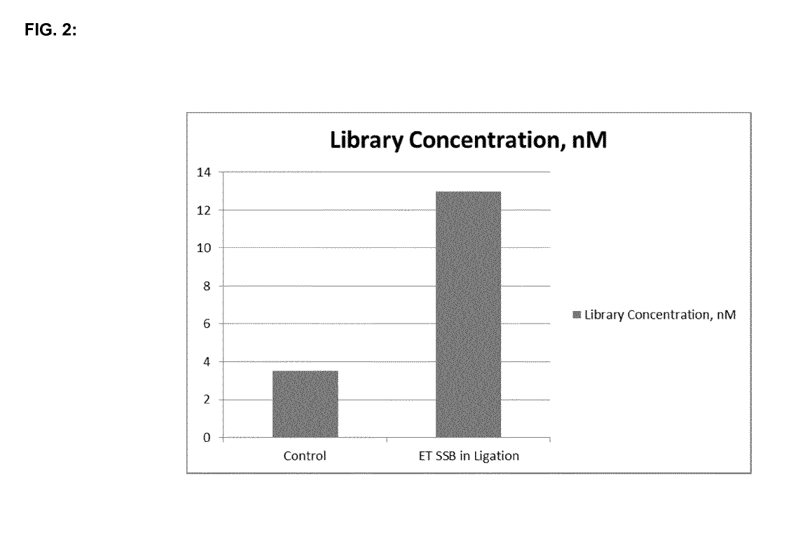

As shown in the FIGS. 1 and 2, both Agilent and qPCR results demonstrated that the addition of the ET SSB into the ligation reaction (‘ET SSB in Ligation’) could significantly increase the library yield and specificity.

### Example 1

The above amplified product of the test and control samples was qualitatively analyzed by using Agilent Bioanalyzer and High Sensitivity DNA Analysis Kit (Agilent).

### Example 2

The above amplified product of the test and control samples was quantitatively analyzed by using qPCR method (QuantiFast Sybr Green Kit, QIAGEN).

1.-14. (canceled)

15. A method of generating a sequencing library for next generation sequencing, the method comprising:
i) providing a genomic template DNA;
ii) mechanically fragmenting the genomic template DNA using a method of ultrasonic acoustic shearing, wherein the acoustic shearing is done with a Covaris instrument and the resulting genomic template DNA fragments are in the size range of between 200 bps and 500 bps, with a median length of about 300 bps;
iii) end-repairing the fragmented DNA with a polynucleotide kinase and a polymerase, wherein the polynucleotide kinase and the polymerase are added simultaneously in one composition, and adding a terminal adenine to the end of the end-repaired DNA fragments by a deoxynucleotidyl enzyme;
iv) ligating cohesive-end adaptors to the end-repaired DNA fragments, wherein the adaptors comprise a 3′-thymidine overhang, thereby generating ligated fragments; and
v) enriching the library by PCR amplification.

16. The method according to claim 1, wherein the polynucleotide kinase is a T4 polynucleotide kinase and the polymerase is a T4 polymerase, and wherein the T4 polymerase has 3′-5′ exonuclease activity.

17. The method according to claim 1, wherein the ligated fragments of step iv) are purified.

18. The method according to claim 1, wherein the ligated fragments are size-selected.

19. A kit comprising:
i) DNA adaptor molecules for use in a ligation reaction for the creation of a Next generation sequencing (NGS) library;
ii) a polymerase with 3′-5′ exonuclease activity; and
iii) a kinase,
wherein the polymerase and the kinase are in one composition.

20. The kit according to claim 19, wherein the polymerase is a T4 polymerase, and the kinase is a T4 kinase, wherein the T4 polymerase and the T4 kinase are in one composition
